(19) United States
(12) Patent Application Publication
(10) Pub. No.: US 2018/0290959 A1
(43) Pub. Date: Oct. 11, 2018

(54) USE OF NOVEL MIXTURES OF (E/Z) CYCLOPENTADECENONE ISOMERS, THE PRODUCTION AND USE THEREOF AS FLAVORING SUBSTANCE

(71) Applicant: BASF SE, Ludwigshafen am Rhein (DE)

(72) Inventors: FRAUKE THRUN, Mannheim (GE); Joaquim Henrique TELES, Waldsee (DE); Albert WERNER, Bishop, TX (US); Raff PELZER, Fürstenberg (DE)

(21) Appl. No.: 15/766,407

(22) PCT Filed: Oct. 7, 2016
(86) PCT No.: PCT/EP2016/074021

§ 371 Date: Apr. 6, 2018

(30) Foreign Application Priority Data
Oct. 8, 2015 (EP) 15188870.8
Feb. 15, 2016 (EP) 16155621.2

(51) Int. Cl. C07C 49/607; C07C 45/54; C07C 45/28; A61Q 13/00; C11B 9/00

(52) U.S. Cl. CPC C07C 49/607; C07C 45/54; C07C 2601/18; A61Q 13/00; C11B 9/0038; C07C 45/28

(57) ABSTRACT

The present invention relates to novel mixtures of cyclopentadecenone isomers, preparation thereof and use thereof as aroma substances, particularly as fragrances, and also aroma substance compositions and agents comprising said mixtures.

The present invention relates to novel mixtures of (E/Z)-cyclopentadecenone isomers, preparation thereof and use thereof as aroma substances, particularly as fragrances, and also aroma substance compositions and agents comprising said mixtures.

### BACKGROUND OF THE INVENTION

In the perfume industry there is a constant demand for new fragrances suitable as fragrance compositions or perfumed articles.

There is a particular demand for musky fragrances and fragrance compositions. This is understood to mean an odor which is similar to the natural musk scent.

Trans- and cis-cyclopentadec-8-enone are already specifically mentioned in U.S. Pat. No. 5,936,100 and in Fürstner, A. et al, Synthesis 1997, 792. Starting from heptadeca-1,16-dienone, the compounds were prepared by means of a ruthenium-catalyzed ring closing alkene metathesis. The use of cis- and trans-cyclopentedec-7-enone as additive in a formulation for inhibiting melanin synthesis is mentioned in EP-A-1 264 594. EP-A 216 185 mentions that the cis-7- and -8-cyclopentadecen-1-one compounds can be prepared via an acid- or Lewis acid-catalyzed Meerwein rearrangement, starting from cis-16-oxabicyclo-[13.1.0]-hexadec-8-ene, without however providing any experimental proof thereof. The usability of both compounds as fragrances is also postulated. The actual olfactory properties of the 7- and 8-cyclopentadecenones have not, however, been described in the literature to date.

The Cu-catalyzed oxidative decarbonylation is known and has been widely published and examples may be found in: a) Tetrahedron Lett. 1969, 12, 985; U.S. Pat. No. 3,496,197; b) Tetrahedron Lett. 1995, 36, 4641, c) Org. Lett. 2011, 13, 2630, d) Bioorg. Med. Chem. Left. 2013, 23, 5949, e) Chin. Chem. Lett. 2014, 25, 771.

Furthermore, the oxidation of cyclohexadeca-1,9-diene with N₂O is known from the prior art (WO 2012/084673). The main product formed in this case is (E/Z)-cyclohexadec-8-enone and also various cyclopentadecenyl carbaldehydes as byproducts. Separation by distillation of the mixture of cyclohexadec-8-enone and cyclopentadecenyl carbaldehyde is only possible with a high loss of yield of cyclohexadec-8-enone. The mixture of cyclohexadec-8-enone and cyclopentadecenyl carbaldehyde must however be separated since otherwise cyclohexadec-8-enone is afflicted with an off-note.

The object of the present invention is to provide novel musky scent substances.

### SUMMARY OF THE INVENTION

Surprisingly, the above object was achieved, in particular, by providing the macrocyclic keto compounds defined in the claims and a method for the preparation and isolation thereof.

It has been found in particular that the cyclic aldehydes, which are formed as byproducts in the oxidation of cyclohexadeca-1,9-diene with nitrous oxide, can be subjected to a Cu-catalyzed oxidative decarbonylation, From the reaction output thus obtained, cyclohexadec-8-enone can be separated by distillation without significant yield losses from the cyclopentadec-8-enones formed and additionally, by means of the distillation, a mixture with valuable olfactory properties can be isolated consisting essentially of (E/Z)-cyclopentadec-8-enone (ratio e.g. 1:1) and (E/Z)cyclopentadec-7-enone (ratio 1:1), in which the ratio of cyclopentadec-8-enone and cyclopentadec-7-enone is 10:1.

It has been found, surprisingly, that the cyclopentadecenones I-IV from the Cu-catalyzed oxidative decarbonylation of cyclopentadecenyl carbaldehyde XI have a very good olfactory smell. The cyclopentadecenones I-IV therefore extend the repertoire of the scent substance industry, particularly the musky fragrances. The compounds are characterized by a green, aldehydic, harsh, gassy, sweet, powdery, dry fruit-like and musk-like smelling odor. In the Cu-catalyzed oxidative decarbonylation, the cyclopentadec-8-enones occur as an (E/Z) mixture in a 1:1 ratio. The (E/Z)-cyclopentadec-7-enones were also obtained as byproducts.

### DETAILED DESCRIPTION OF THE INVENTION

### a) General Definitions

An “aroma chemical” is a generic term for compounds which may be used as “fragrance” and/or as “flavoring”.

In the context of the present invention, “fragrance” is understood to mean natural or synthetic substances having intrinsic odor.

In the context of the present invention, “flavoring” is understood to mean natural or synthetic substances having intrinsic flavor.

In the context of the present invention, “odor” or “olfactory perception” is the interpretation of the sensory stimuli which are sent from the chemoreceptors in the nose or other olfactory organs to the brain of a living being. The odor can be a result of sensory perception by the nose of fragrances, which occurs during inhalation. In this case, the air serves as odor carrier.

In the context of the present invention, “scent” is understood to mean a pleasant-smelling odor, The same applies to a “scent substance” according to the invention.

In the context of the present invention, a “perfume” is a mixture of fragrances and carriers such as, in particular, an alcohol.

In the context of the present invention, a “perfume composition” is a perfume comprising different amounts of individual components harmoniously balanced with one another. The properties of the individual constituents are employed in order to achieve a new overall image in the combination, wherein the characteristics of the ingredients retire into the background but without being suppressed.

In the context of the present invention, a “perfume oil” is a concentrated mixture of several fragrances which are employed, for example, in alcoholic solutions, for perfuming different products.

In the context of the present invention, a “scent theme” is the prevailing scent note in a fragrance composition.

In the context of the present invention, the “top note” is the first phase of the scent progression of a perfume. It plays the decisive role during the first impression upon opening the bottle and while applying the perfume to the skin. The aim of the top note is to arouse interest in the perfume generally and to ensure attention. Consequently, an extraordinary character is often more important than a polished harmony. The top note is naturally determined by readily volatile fragrances.

In the context of the present invention, “modifying” signifies that the basic theme of a fragrance composition is provided with additional or different accords and odor nuances.

In the context of the present invention, “accords” are formed by combining different fragrances, which thus combine to give new odor images. The number of fragrances used can range from two up to a hundred.

In the context of the present invention, an “organoleptically/sensorily effective amount” of a fragrance is the amount which suffices to produce a stimulatory effect on a sensory organ or sensory receptor.

Unless otherwise stated, (E/Z) represents (E and/or Z) and in principle refers to mixtures comprising both the E-configuration and the corresponding Z-configuration stereoisomers and also the stereoisomerically pure E and Z forms of a compound.

### b) Special Embodiments of the Invention

The present invention relates especially to the following subjects:
1. A method for preparing at least one macrocyclic, particularly monocyclic macrocyclic keto compound of the general formula X

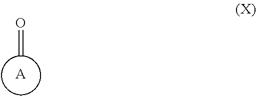

wherein A is a cycloaliphatic hydrocarbon residue having m ring carbon atoms, where m is an integer from 13 to 17, e.g. 13, 14, 15, 16 or 17, in particular 15, and optionally has n C═C double bonds, where n is an integer equal to 1, 2 or 3, particularly 1

wherein

a) at least one cycloaliphatic carbaldehyde compound of formula XI

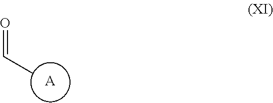

where A is as defined above, is oxidatively decarbonylated; wherein the compound of formula XI is reacted in the presence of an amount, preferably a catalytic amount, of a preferably homogeneous Cu(II) catalyst and pure molecular oxygen, lean air or preferably air, particularly dried, filtered ambient air and optionally

b) at least one compound of formula X is isolated from the reaction mixture.

The target compound of formula X is obtained in this case in stereoisomerically pure form or in the form of stereoisomeric mixtures comprising at least 2, in particular 2, 3 or 4, stereoisomeric forms of such keto compounds; or wherein substance mixtures are obtained comprising at least 2, e.g. 2, 3, or 4, particularly 2 or 3, keto compounds of this type, in each case in stereoisomerically pure form or as a stereoisomeric mixture. In particular, compounds are preferably obtained in which, in the case where n=1, the keto group and the ring C═C double bond are 4 to 7 ring carbon atoms distant from each other. Substance mixtures may also comprise constitutional isomers when n is not 0. If n is 2 or 3, then the double bonds are not cumulative.
2. The method according to embodiment 1, wherein the oxidative decarbonylation is carried out additionally in the presence of an in particular organic base.

The base used, for example, in the method according to the invention is, for example, selected from diazabicycloalkanes such as diazabicyclooctane (DABCO), diazabicycloundecene (DBU), diazabicyclononane (DBN) tertiary amines such as trimethylamine, triethylamine, triisopropylamine, diisopropylethylamine or tripropylamine, N,N-dimethylpiperazine, N-methylpyridine, N-methylpyrrolidone, quinuclidine and the like. Preference is given to using DBU.
3. The method according to any of the preceding embodiments, wherein the oxidative decarbonylation is carried out in the absence or in the presence of a solvent, such as in the presence of an organic solvent, particularly an organic solvent, in which the catalyst is dissolved.

Non-limiting examples of organic solvents include: polar, aprotic solvents such as dimethylformamide (DMF), hexamethylphosphoramide (HMPA), dimethyl sulfoxide (DMSO), tetramethylurea and dimethylacetamide, and also mixtures thereof. Further suitable organic solvents, which can be used alone or in combination with the above aprotic organic solvents, are alcohols such as methanol, ethanol, propanol or butanols such as tertbutanol and also tetrahydrofuran, dioxane or benzene. Preference is given to using DMF.

In an alternative embodiment, the reaction is carried out essentially without addition of solvent, preferably in a solvent-free reaction mixture. The mixture of reactants and catalyst may be heated for this purpose, for example to a temperature of 30 to 70° C. or 40 to 60° C.
4. The method according to any of the preceding embodiments, wherein the catalyst is formed in situ by adding an in particular bidentate ligand, preferably a diamine ligand, to a Cu(II) salt.

The Cu(II) salt used according to the invention is selected, for example, from Cu(II) acetate, formate, sulfate, chloride or nitrate. Preference is given to using Cu(OAc)₂.

Suitable complexing ligands are particularly bidentate Cu-complexing amine ligands such as N,N,N′,N′-tetramethylethylenediamine (TMEDA), 1,10-phenanthroline and 2,2′-bipyridyl. Preference is given to using TMEDA.
5. The method according to any of the preceding embodiments, wherein the oxidative decarbonylation is carried out at a temperature in the range of 10 to 70, particularly 30 to 60° C., over a period of 0.1 to 40, particularly 0.1 to 30 h, preferably 2 to 7 h, above all 3 to 5 h, e.g. approximately 4 h, and at a pressure of 0.1 to 10, particularly 1 to 2 atm. It is also feasible to carry out the method under reduced pressure, for example, at a reduced pressure in the range of 0.001 to 0.99 bar, or particularly 0.01 to 0.5 or 0.1 to 0.25 bar.
6. The method according to any of the preceding embodiments, wherein m is 15 and/or n is 1, in particular m is 15 and n is 1, in the compounds of formula X and XI.
7. The method according to any of the preceding embodiments, wherein (E/Z)cyclopentadec-8-enyl-1-carbaldehyde or a substance mixture comprising this compound, such as a mixture of (E/Z)-cyclopentadec-8-enyl- and (E/Z)-cyclopentadec-7-enyl-1-carbaldehyde, is used.
8. The method according to embodiment 7, wherein at least one compound is obtained selected from (E/Z)-cyclopentadec-8-en-1-one and (E/Z)-cyclopentadec-7-en-1-one.
9. The method according to embodiment 8, wherein at least one compound is obtained selected from compounds of the following formulae I, II, III and IV,

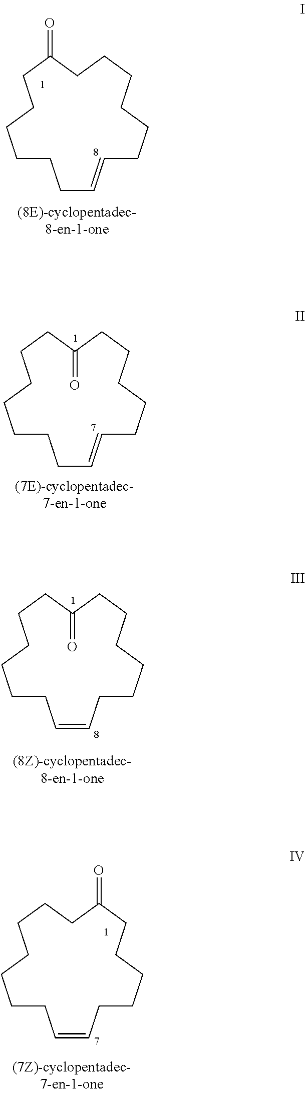 [SMILES: O=C1CCCCCC/C=C/CCCCCC1 ; O=C1CCCCC/C=C/CCCCCCC1 ; O=C1CCCCCC/C=C\CCCCCC1 ; O=C1CCCCC/C=C\CCCCCCC1]

in particular a substance mixture essentially (i.e. to at least 80% or 90% (i.e. area %) composed of the compounds I and III or a substance mixture essentially (i.e. to at least 80% or 90% (area %) composed of the compounds I, II and III or composed of the compounds I, II and III and optionally IV.
10. The method according to any of the preceding embodiments, wherein a reaction product is used in stage a) obtained from the dinitrogen monoxide oxidation of a cycloaliphatic compound of formula XII

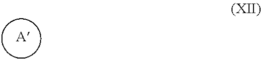

wherein A′ is a cycloaliphatic hydrocarbon residue having m+1 ring carbon atoms, where m is an integer from 13 to 17, e.g. 13, 14, 15, 16 or 17, in particular 15, and optionally has n+1 C═C double bonds, where n is an integer equal to 1, 2 or 3, particularly 1.
11. The method according to embodiment 10, wherein m is 15 and n is 1 in the compound of formula XII.
12. The method according to embodiment 11, wherein the compound of formula XII is cyclohexadeca-1,9-diene.
13. A method for preparing Globanone ((E/Z)-cyclohexadec-8-en-1-one), wherein
   a) cyclohexadeca-1,9-diene is oxidized with dinitrogen monoxide, wherein a reaction mixture is obtained which comprises a mixture, comprising (E/Z)-cyclohexadec-8-en-1-one (Globanone) and at least one cyclopentadecenyl carbaldehyde compound, particularly selected from (E/Z)-cyclopentadec-8-enyl carbaldehyde; wherein unreacted cyclohexadeca-1,9-diene is optionally removed, for example, by distillation, and this is then optionally fed back into the method, stage a);
   b) the reaction mixture from stage a) is subjected to an oxidative decarbonylation reaction according to any of embodiments 1 to 5; and
   c) Globanone is separated from the cyclopentadecenones thus formed, particularly the cyclopentadecenones I, II, III and/or IV thus formed or mixtures thereof.

The oxidative decarbonylation can take place in this case in the presence or absence of a solvent.
14. The method according to embodiment 13, wherein the separation in stage c) is carried out by distillation.
15. A substance or substance mixture comprising at least one macrocyclic keto compound of the general formula X above.
16. The substance mixture according to embodiment 15 comprising essentially (i.e. to at least 80% or 90% (area %) at least two compounds selected from (E/Z)-cyclopentadec-8-en-1-one and (E/Z)-cyclopentadec-7-en-1-one.
17. The substance mixture according to embodiment 16 comprising essentially (i.e. to at least 80% or 90% (area %) at least two compounds selected from compounds of the following formulae I, Ill and the regioisomers thereof, such as of the formula II

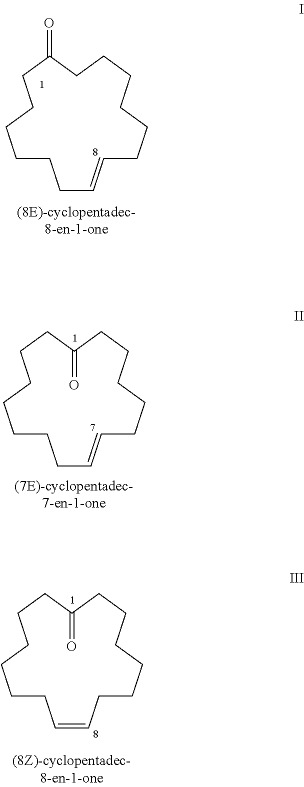 [SMILES: O=C1CCCCCC/C=C/CCCCCC1 ; O=C1CCCCC/C=C/CCCCCCC1 ; O=C1CCCCCC/C=C\CCCCCC1]

or comprising essentially (i.e. to at least 80% or 90% (area %) at least two compounds selected from compounds of the following formulae I and Ill and the regioisomers thereof, such as of the formulae II and IV

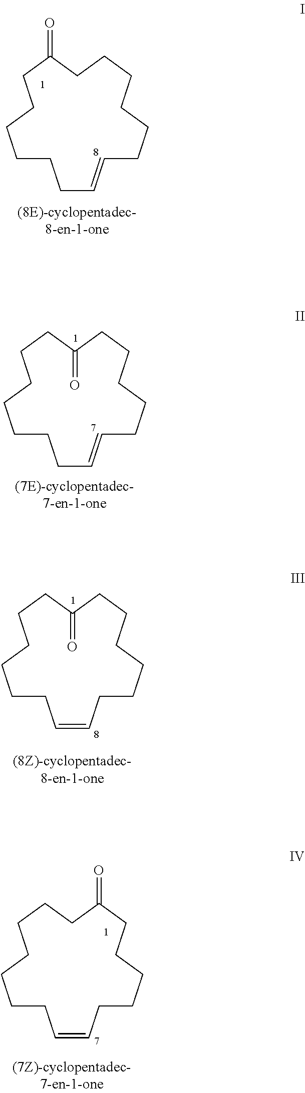 [SMILES: O=C1CCCCCC/C=C/CCCCCC1 ; O=C1CCCCC/C=C/CCCCCCC1 ; O=C1CCCCCC/C=C\CCCCCC1 ; O=C1CCCCC/C=C\CCCCCCC1]

18. The use of at least one substance or substance mixture according to any of embodiments 15 to 17 as an aroma chemical, particularly as a fragrance.
19. The use according to embodiment 18 in agents selected from perfumes, washing and cleaning compositions, cosmetic agents, body care agents, hygiene articles, food, food supplements, scent dispensers, fragrances, pharmaceutical agents and plant protection agents.
20. The use according to either of embodiments 18 and 19 of a mixture essentially comprising (E)-cyclopentadec-8-en-1-one (I) and (Z)-cyclopentadec-8-en-1-one (III).
21. The use according to embodiment 20, wherein the molar ratio of (I) to (III) is in the range of approximately 1:1 to approximately 10:1, particularly 1:1 to 5:1.
22. The use according to any of embodiments 18 to 21 for generating a musk note in a fragrance composition.
23. The use according to embodiment 22, to impart, modify and/or enhance a musk scent note in a fragrance composition by admixing a sensorily effective amount of at least one substance or one substance mixture according to the definition in any of embodiments 15 to 17.
24. A fragrance composition comprising at least one substance or one substance mixture according to the definition in any of embodiments 15 to 17 or prepared by one of the methods according to embodiments 1 to 12.
25. A composition according to embodiment 21, comprising the substance or the substance mixture in a proportion by weight of 0.01 to 99.9% by weight, 1 to 80% by weight, 2 to 50% by weight, 3 to 25 or 5 to 15% by weight, based on the total weight of the composition.
26. An agent comprising at least one substance or one substance mixture according to the definition in any of embodiments 15 to 17 or prepared by one of the methods according to embodiments 1 to 12.
27. The agent according to embodiment 26, comprising the substance or the substance mixture in a proportion by weight of 0.01 to 99.9% by weight, 1 to 80% by weight, 2 to 50% by weight, 3 to 25 or 5 to 15% by weight, based on the total weight of the agent.
28. The agent according to embodiment 26 or 27, selected from perfumes, washing and cleaning compositions, cosmetic agents, body care agents, hygiene articles, food, food supplements, scent dispensers, fragrances, pharmaceutical agents and plant protection agents.

### c) Further Configurations of the Invention

### c1) Fragrance Compositions:

According to a further aspect, the fragrances used in accordance with the invention are used particularly for the purpose of efficient handling and dosing, and as fragrance mixtures with diluents or solvents. In this case, the proportion of fragrances, based on the sum total of fragrances and solvent, is given in % by weight.

Solvent:

In the context of the present invention, a “solvent” serves as the diluent of the fragrances to be used according to the invention or the fragrance composition according to the invention but without having any intrinsic odorous properties. Some solvents also have fixing properties.

The compound of formula X according to the invention, or a substance mixture defined above composed of several compounds/isomers of formula X, can be added to 0.1 to 99% by weight to a diluent or solvent. Preference is given to at least 40% by weight solutions, more preferably at least 50% by weight solutions, further preferably at least 60% by weight solutions, more preferably at least 70% by weight solutions, particularly preferably at least 80% by weight solutions, especially preferably at least 90% by weight solutions, preferably in olfactory acceptable solutions.

Preferred olfactorily acceptable solvents are ethanol, isopropanol, dipropylene glycol (DPG), propylene glycol, 1,2-butylene glycol, glycerol, diethylene glycol monoethyl ether, diethyl phthalate (DEP), isopropyl myristate (IPM), triethyl citrate (TEC), benzyl benzoate (BB) and benzyl acetate. In this case, preference is given in turn to ethanol, diethyl phthalate, propylene glycol, dipropylene glycol, triethyl citrate, benzyl benzoate and isopropyl myristate.

In the context of the present invention, a “fragrance composition” is a mixture which, in addition to a compound of formula X according to the invention or a substance mixture of two or more compounds/isomers of formula X defined above, comprises at least one further fragrance. Such a fragrance composition may particularly take the form of a perfume composition (a perfume oil).

Fragrance compositions according to the invention comprise, for example, an amount of a compound of formula X according to the invention, or a substance mixture of two or more compounds/isomers of formula X defined above, of 0.01 to 65% by weight, preferably of approximately 0.1 to approximately 50% by weight, preferably of approximately 0.5 to approximately 30% by weight and particularly preferably of approximately 0.5 to approximately 25% by weight, based on the total amount of the fragrance composition. The ratio by weight of compound(s) of formula X according to the invention to the total amount of further fragrances is in the range, for example, of 1:1000 to 1:0.5, preferably in the range of 1:700 to 1:1, particularly preferably in the range of 1:500 to 1:10.

Fragrance compositions according to the invention comprise, for example, an amount of compound(s) of the formula X according to the invention of 0.01 to 65% by weight and preferably of approximately 0.1 to approximately 50% by weight, preferably of approximately 0.5 to approximately 30% by weight and particularly preferably of approximately 0.5 to approximately 25% by weight, based on the total amount of the fragrance composition. The ratio by weight of compound(s) of formula X according to the invention to the total amount of further (different therefrom) fragrances is in the range, for example, of 1:1000 to 1:0.5, preferably in the range of 1:700 to 1:1, particularly preferably in the range of 1:500 to 1:10.

The data above on the content of the compounds of formula X also apply correspondingly to the preferred compounds of the formulae I, II, III and IV, particularly also to substance mixtures of the compounds of the formulae I, II, III and IV.

### Further Fragrances:

Fragrance compositions according to the invention, in addition to the compound(s) of formula X according to the invention (in particular the formulae I, II and III), comprise at least one further fragrance, preferably 2, 3, 4, 5, 6, 7, 8 or more further fragrances, in which further fragrances are selected from, for example:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis-isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styralyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyl-oct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), 7-methyl-2H-1,5-benzodioxepin-3(4H)-one (Calone¹⁹⁵¹⁵), 3,3,5-trimethylcyclohexyl acetate (preferably with a content of cis-isomers of 70% by weight) or more and 2,5,5-trimethyl-1,2,3,4,4a,5,6,7-octahydronaphthalen-2-ol (Ambrinol S¹). In the context of the present invention, the fragrances mentioned above are accordingly preferably combined with mixtures according to the invention. ¹ Trade name of Symrise GmbH, Germany;² Trade name of Givaudan A G, Switzerland;³ Trade name of International Flavors & Fragrances Inc., USA;⁵ Trade name of Danisco Seillans S. A., France;⁹ Trade name of Firmenich S. A., Switzerland;¹⁰ Trade name of PFW Aroma Chemicals B.V., The Netherlands.

If trade names are specified above, these refer to the following sources:

Further fragrances with which (E/Z)-cyclopentadec-7/8-enone may be combined, for example, to give a fragrance composition are found, for example, in S. Arctander, Perfume and Flavor Chemicals, Vol. I and II, Montclair, N.J., 1969, Author's edition or K. Bauer, D. Garbe and H. Surburg, Common Fragrance and Flavor Materials, 4th. Ed., Wiley-VCH, Weinheim 2001. Specific examples are:

extracts from natural raw materials such as essential oils, concretes, absolutes, resins, resinoids, balsams, tinctures such as e.g.

ambergris tincture; amyris oil; angelica seed oil; angelica root oil; aniseed oil; valerian oil; basil oil; tree moss absolute; bay oil; lungwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oak moss absolute; elemi oil; tarragon oil; eucalyptus citriodora oil; eucalyptus oil; fennel oil; spruce needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; ylang ylang oil; hyssop oil; civet absolute; cinnamon leaf oil; cinnamon bark oil, and fractions thereof, or ingredients isolated therefrom;

individual fragrances from the group of hydrocarbons, such as e.g. 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof such as e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;

the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

the aliphatic nitriles such as e.g. 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;

the esters of aliphatic carboxylic acids such as e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl-2,4-decadienoate; methyl 2-octinate; methyl 2-noninate; allyl 2-isoamyloxy acetate; methyl-3,7-dimethyl-2,6-octadienoate; 4-methyl-2-pentyl crotonate;

the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;

the acyclic terpene aldehydes and ketones such as e.g. geranial; neral; citronellal; 7-hydroxy-3,7-dimethyloctanal; 7-methoxy-3,7-dimethyloctanal; 2,6,1-trimethyl-9-undecenal; geranyl acetone; as well as the dimethyl and diethyl acetals of geranial, neral, 7-hydroxy-3,7-dimethyloctanal; the cyclic terpene alcohols such as e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;

the cyclic terpene aldehydes and ketones such as e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8-(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);

the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

the cycloaliphatic alcohols such as e.g. alpha-3,3-trimethylcyclohexylmethanol; isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

the cyclic and cycloaliphatic ethers such as e.g. cineol; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;

the cyclic and macrocyclic ketones such as e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-yl-cyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tertpentylcyclohexanone; cyclohexadec-5-en-1-one; 6,7-dihydro-1,1,2,3,3-pentamethyl-4(5H)indanone; 8 cyclohexadecen-1-one; 7-cyclohexadecen-1-one; (7/8)-cyclohexadecen-1-one; 9 cycloheptadecen-1-one; cyclopentadecanone; cyclohexadecanone;

the cycloaliphatic aldehydes such as e.g. 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

the cycloaliphatic ketones such as e.g. 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl(2,4-dimethyl-3-cyclohexen-1-yl) ketone;

the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7-methanooctahydro-5 or 6-indenyl acetate;

the esters of cycloaliphatic alcohols such as e.g. 1-cyclohexylethyl crotonate;

the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;

the araliphatic alcohols such as e.g. benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;

the araliphatic ethers such as e.g. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]m-dioxine; 4,4a,5,9b-tetrahydro-2,4-dimethylindeno[1,2-d]-m-dioxine;

the aromatic and araliphatic aldehydes such as e.g. benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

the aromatic and araliphatic ketones such as e.g. acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2 naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tertbutyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

the aromatic and araliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenyiglycidate; ethyl 3-methyl-3-phenylglycidate;

the nitrogen-containing aromatic compounds such as e.g. 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentanonitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;

the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

the heterocyclic compounds such as e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

### c2) Fragrance-Containing Articles

(E/Z)-cyclopentadec-718-enone according to the invention or fragrance compositions according to the invention can be incorporated into a series of products or applied to said products.

Fragrances according to the invention can be used in the production of perfumed articles. The olfactory properties, like the material properties (such as solubility in customary solvents and compatibility with further customary constituents of such products), as well as the toxicological acceptability of the fragrances according to the invention underline their particular suitability for the stated use purposes. The positive properties contribute to the fact that the fragrances used according to the invention and the fragrance compositions according to the invention are particularly preferably used in perfume products, body care products, hygiene articles, textile detergents and in cleaners for solid surfaces.

The perfumed article is e.g. selected from perfume products, body care products, hygiene articles, textile detergents and cleaners for solid surfaces. Preferred perfumed articles according to the invention are also selected from among:

perfume products selected from perfume extracts, Eau de Parfums, Eau de Toilettes, Eau de Colognes, Eau de Solide, Extrait Parfum, air fresheners in liquid form, gel-like form or a form applied to a solid carrier, aerosol sprays, scented cleaners and oils;

body care products selected from aftershaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics such as e.g. eye shadows, nail varnishes, make-ups, lipsticks, mascara, toothpaste, dental floss;

hygiene articles selected from candles, lamp oils, joss sticks, insecticides, repellents, propellants, rust removers, perfumed freshening wipes, armpit pads, baby diapers, sanitary towels, toilet paper, cosmetic wipes, pocket tissues, dishwasher deodorizer;

cleaners for solid surfaces selected from perfumed acidic, alkaline and neutral cleaners, such as e.g. floor cleaners, window cleaners, dishwashing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, lime scale removers, grill and oven cleaners, algae and moss removers, mold removers, facade cleaners;

textile detergents selected from liquid detergents, powder detergents, laundry pretreatments such as bleaches, soaking agents and stain removers, fabric softeners, washing soaps, washing tablets.

According to a further aspect, the fragrances used according to the invention and the fragrance compositions according to the invention are suitable for use in surfactant-containing perfumed articles. This is because fragrances and/or fragrance compositions with a rose top note and pronounced naturalness are often sought—especially for the perfuming of surfactant-containing formulations such as, for example, cleaners (in particular dishwashing compositions and all-purpose cleaners).

According to a further aspect, fragrances used according to the invention and fragrance compositions according to the invention can be used as agents for providing (a) hair or (b) textile fibers with a rosy odor note.

The fragrances to be used according to the invention and fragrance compositions according to the invention are therefore particularly well suited for use in surfactant-containing perfumed articles.

It is preferred if the perfumed article is one of the following:
an acidic, alkaline or neutral cleaner which is selected in particular from the group consisting of all-purpose cleaners, floor cleaners, window cleaners, dishwashing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish, which is selected in particular from the group consisting of furniture polishes, floor waxes and shoe creams, or
a body care composition, which is selected in particular from the group consisting of shower gels and shampoos shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semipermanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics.

Ingredients with which fragrances used according to the invention or fragrance compositions according to the invention can preferably be combined are, for example: preservatives, abrasives, antiacne agents, agents to combat skin aging, antibacterial agents, anticellulite agents, antidandruff agents, anti-inflammatory agents, irritation-preventing agents, irritation-alleviating agents, antimicrobial agents, antioxidants, astringents, sweat-inhibiting agents, antiseptics, antistatics, binders, buffers, carrier materials, chelating agents, cell stimulants, cleaning agents, care agents, hair removal agents, surface-active substances, deodorizing agents, antiperspirants, emollients, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfolisting agents, silicones, skin-calming agents, skin-cleansing agents, skin care agents, skinhealing agents, skin lightening agents, skin-protective agents, skin-softening agents, cooling agents, skin-cooling agents, warming agents, skin-warming agents, stabilizers, UV-absorbent agents, UV filters, detergents, fabric softeners, suspending agents, skin-tanning agents, thickeners, vitamins, oils, waxes, fats, phospholipids, saturated fatty acids, mono or polyunsaturated fatty acids, a-hydroxy acids, polyhydroxy fatty acids, liquefiers, dyes, color-protection agents, pigments, anticorrosives, aromas, flavorings, fragrances, polyols, surfactants, electrolytes, organic solvents or silicone derivatives.

According to a further aspect, the fragrances are used in the production of the perfumed articles in liquid form, undiluted or diluted with a solvent or in the form of a fragrance composition. Suitable solvents for this purpose are e.g. ethanol, isopropanol, diethylene glycol monoethyl ether, glycerol, propylene glycol, 1,2-butylene glycol, dipropylene glycol, diethyl phthalate, triethyl citrate, isopropyl myristate, etc. If the specified solvents have their own olfactory properties, they are assigned exclusively to the constituent “solvent” and not to the “fragrances”.

The fragrances and/or fragrance compositions present in the perfumed articles according to the invention can in this connection, in one embodiment, be absorbed onto a carrier, which ensures both fine distribution of the fragrance or fragrance composition within the product and also controlled release upon use, Carriers of this type may be porous inorganic materials such as light sulfate, silica gels, zeolites, gypsums, clays, clay granules, aerated concrete, etc. or organic materials such as woods and cellulose-based materials.

The fragrances used according to the invention and the fragrance compositions according to the invention can also be in microencapsulated form, spray-dried form, in the form of inclusion complexes or in the form of extrusion products and be added in this form to the product or article to be perfumed. The properties can be further optimized by so-called “coating” with suitable materials with regard to a more targeted release of the scent, for which purpose preferably waxy synthetic substances such as e.g. polyvinyl alcohol are used.

The microencapsulation can take place for example by the so-called coacervation method with the help of capsule materials, e.g. made of polyurethane-like substances or soft gelatin, The spray-dried perfume oils can be produced for example by spray-drying an emulsion or dispersion comprising the perfume oil, wherein carrier substances that can be used are modified starches, proteins, dextrin and vegetable gums. Inclusion complexes can be prepared e.g. by introducing dispersions of fragrance compositions and cyclodextrins or urea derivatives into a suitable solvent, e.g. water. Extrusion products can be prepared by melting fragrances used according to the invention and fragrance compositions according to the invention with a suitable wax-like substance and by extrusion with subsequent solidification, optionally in a suitable solvent, e.g. isopropanol.

### c3) Preparation of Fragrances According to the Invention

The macrocyclic ketones of the formula X according to the invention are prepared starting from the corresponding carbaldehydes of formula XI

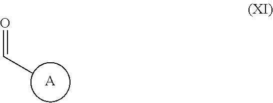

wherein A is a cycloaliphatic hydrocarbon residue having m ring carbon atoms, where m is an integer from 13 to 17, and optionally has n C═C double bonds, where n is an integer equal to 1, 2 or 3.

The carbaldehydes in turn are prepared, for example, starting from the corresponding cycloaliphatic compounds of the formula XII

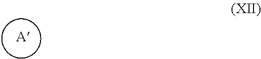

where A′ is a cycloaliphatic hydrocarbon residue having m+1 ring carbon atoms, where m is an integer from 13 to 17, and optionally has n+1 C═C double bonds, and where n is an integer equal to 1, 2 or 3, and is accessible by dinitrogen monoxide oxidation.

### i) Synthesis of Carbaldehyde (XI) by N₂O Oxidation

As described, for example, by the applicant in PCT/EP2015/072544.

Starting from the corresponding aliphatic carbocycle, in particular a cyclic mono- or polyunsaturated olefin XII (i.e. in comparison to the cyclic residue A in the product of formula X, the starting compound XII comprises an additional C═C double bond and an additional ring carbon atom), compounds of formula XI are accessible by dinitrogen monoxide oxidation, as described for example in WO 2012/084673.

An example of a suitable starting compound (which can be used both in stereoisomerically pure form and in the form of stereoisomeric mixtures) for preparing compounds of formula XI, where A is a monounsaturated C15 residue, is cyclohexadeca-1,9-diene which is either obtainable commercially or may be prepared according to Example 2 in EP-A-1 288 181.

In particular in this case, a cyclic olefin is oxidized by reaction with dinitrogen monoxide. Dinitrogen monoxide may be used here in pure form or optionally diluted with other substances gaseous under the reaction conditions, such as carbon dioxide.

The reaction of the cyclic olefin with dinitrogen monoxide can be carried out without solvent or in the presence of at least one suitable solvent or diluent. The reaction is preferably carried out in the absence of solvent. All customary solvents and/or diluents are essentially suitable here, but with the proviso that they neither have a C═C double bond nor a C—C triple bond, nor an aldehyde group. Suitable solvents to be mentioned include, inter glia: cyclic alkanes, for example, cyclohexane, cyclopentane, cyclooctane, cyclododecane or saturated aliphatic or aromatic, optionally alkyl-substituted hydrocarbons.

The temperature in the reaction is, for example, from 140 to 350° C., in particular from 180 to 320° C. or from 200 to 300° C. It is also possible to carry out the reaction at two or more temperatures or in two or more temperature ranges which are in each case within the limits specified above. Temperature changes in the course of the reaction may be implemented continuously or discontinuously. However, the reaction temperature is in particular essentially constant. However, the reaction may also preferably be carried out adiabatically, such that the temperature increases in the reactor.

The pressure during the reaction of the cyclic olefin with dinitrogen monoxide is in particular higher than the autogenous pressure of the reactant or product mixture at the selected reaction temperature(s). The pressure is, for example, from 1 to 1000 bar, such as from 40 to 300 bar or from 50 to 200 bar.

It is possible to carry out the reaction of the cyclic olefin with dinitrogen monoxide at two or more pressures or in two or more pressure ranges which are in each case within the limits specified above. Pressure changes in the course of the reaction may be implemented continuously or discontinuously. However, the pressure during the reaction is in particular essentially constant.

With regard to the reactors which can be used for the reaction (on a laboratory or production scale), there are no particular limitations. In particular, the reaction may be carried out in batch mode or in continuous mode. Consequently, the reactors used may be, for example, at least one CSTR (continuous stirred tank reactor) with at least one internal and/or at least one external heat exchanger, at least one tubular reactor, at least one tube bundle reactor or at least one loop reactor. It is also possible to configure at least one of these reactors such that it has at least two different zones. Such zones may, for example, differ in reaction conditions such as, for example, the temperature or the pressure and/or in the geometry of the zone such as, for example, the volume or the cross section. If the reaction is carried out in two or more reactors, two or more identical reactor types or at least two different reactor types may be used. In particular, the reaction with dinitrogen monoxide is carried out in a single reactor. For example, the reaction may be carried out in batch mode or in continuous mode.

The residence time of the reaction mixture in the reactor is generally in the range from 0.1 to 40 hours, preferably in the range from 0.1 to 30 hours, more preferably in the range from 2 to 7 hours.

In the feed, the molar ratio of dinitrogen monoxide to the cyclic olefin is generally in the range from 0.01 to 30, for example in the range from 0.03 to 10, particularly preferably in the range from 0.05 to 1 and especially preferably in the range from 0.08 to 0.2.

Since dinitrogen monoxide is preferably used in deficiency, only a portion of the olefin XII (e.g. cyclohexadeca-1,9-diene) is reacted. Unreacted olefin (e.g. cyclohexadeca-1,9-diene) is separated from the reaction product by distillation and fed back again to the reaction. In this case, the unreacted olefin (e.g. cyclohexadeca-1,9-diene) is the overhead product and the reaction product is the bottoms product of the column. The distillation in this case is conducted, for example, at a pressure at the top of 20 mbar and, for example, a bottom temperature of 210° C. The differential pressure across the column is, for example, 18 mbar. The column is equipped, for example, with a structured fabric packing of the Montz A3 type. The packing height is, for example, 4 m and the feed is, for example, 2 m. From the bottoms output obtained, the compound XI is then isolated as a secondary component by distillation.

The desired reaction product XI is formed here as a secondary component such that the reaction mixture must be purified in a suitable manner.

However, in a further variant of the invention, the direct reaction of the reaction mixture to the desired ketone of formula X is carried out without any further purification. However, an additional purification can also be carried out.

In the case of a purification, this can be carried out, for example, by distillation (such as in particular by fractional distillation, preferably under reduced pressure) or by chromatography. Suitable purification methods are familiar to those skilled in the art. The purification may be carried out, for example, in batch mode or continuously.

For example, the distillation by means of a distillation column may use packings known to those skilled in the art. The optimal distillation conditions can be established by those skilled in the art without undue effort. The distillation can be carried out in particular under vacuum, for example at a pressure of <1000 mbar, <500 mbar, <300 mbar, <100 mbar or <10 mbar. The distillation column used may have several, for example, at least 20, at least 25 or at least 30, such as up to 70 theoretical plates. The reflux ratio can be, for example, in the range of about 5 to 100 and be at least 20, at least 25 or at least 30 and is in particular about 100 for a particularly advantageous fractionation.

Column chromatography can also be carried out, for example, in place of or in addition to purification by distillation. In this case, column materials and mobile phases known to those skilled in the art are used. Optimal chromatography conditions, such as column geometry and mobile phase flow rate, can be established by those skilled in the art without undue effort.

Examples of suitable column materials are polar adsorbents such as iron oxide Fe₂O₃, aluminum oxide, carbohydrates or silica gel, with or without additives such as fluorescence indicators or gypsum.

Examples of suitable mobile phases are: aliphatic or aromatic mobile phases such as alkanes or cycloalkanes, for example pentane, petroleum ether, hexane, heptane, toluene or the corresponding cyclic compounds; aliphatic ethers, esters or, for example, Et₂O, MTBE, EtOAc, acetone or mixtures of such mobile phases such as hexane/MTBE, hexane/EtOAc, pentane/Et₂O, petroleum ether/Et₂O.

In this case, a desired carbaldehyde of formula X, or mixtures thereof, may be isolated in pure form or at a purity of more than 20, for example, more than 30, more than 40, more than 50, more than 60, more than 70 or more than 80% by weight.

The carbaldehyde of formula XI can be isolated here in stereoisomerically pure form, or in particular as a mixture of two or more stereoisomers, particularly if residue A has a C═C double bond.

In particular, trans-cyclopentadec-8-enyl-1-carbaldehyde and/or cis-cyclopentadec-8-enyl-1-carbaldehyde are accessible in this manner.

### ii) Synthesis of Cyclic Ketone (X) by Oxidative Decarbonylation

Starting from the corresponding cyclic carbaldehyde of general formula (XI) (obtainable purified or as a reaction mixture as described above), the oxidative decarbonylation is carried out according to Cu(II)-based decarbonylations known from the prior art, (cf e.g. a) Tetrahedron Lett. 1969, 12, 985; U.S. Pat. No. 3,496,197; b) Tetrahedron Lett. 1995, 36, 4641, c) Org. Lett. 2011, 13, 2630, d) Bioorg. Med. Chem. Lett. 2013, 23, 5949, e) Chin. Chem. Lett. 2014, 25, 771).

A specific, non-limiting example of a suitable starting compound (which can be used both in stereoisomerically pure form and in the form of stereoisomeric mixtures) for preparing compounds of formula X, where A is a monounsaturated C₁₅ residue, is cyclopentadeca-8-enyl carbaldehyde and cyclopentadeca-7-enyl carbaldehyde.

The catalytic reaction of the cyclic carbaldehydes with molecular oxygen according to the invention takes place optionally in the presence of at least one suitable solvent or diluent.

In this case, oxygen can be used in the process as pure oxygen or preferably as a constituent of ambient air or lean air.

Examples of suitable solvents include, inter alia: polar, aprotic solvents such as dimethylformamide (DMF), hexamethylphosphoramide (HMPA), dimethyl sulfoxide (DMSO), tetramethylurea and dimethylacetamide, and also mixtures thereof. Further suitable organic solvents, which can be used alone or in combination with the above aprotic organic solvents, are alkanols such as methanol, ethanol, propanol, isopropanol or butanols such as tert-butanol and also tetrahydrofuran, dioxane or benzene.

The catalytic reaction of the cyclic carbaldehydes can also take place in the absence of a solvent or diluent.

Cu(II)-based catalysts, particularly homogeneous catalysts, are used as catalyst. These are preferably formed in situ in the reaction mixture by adding an in particular bidentate ligand, preferably a diamine ligand, to a Cu(II) salt.

The Cu(II) salt used according to the invention is selected, for example, from Cu(II) acetate, formate, sulfate, chloride or nitrate. Preference is given to using Cu(OAc)₂.

Suitable complexing ligands are particularly bidentate copper-complexing amine ligands such as N,N,N′,N′-tetramethylethylenediamine (TMEDA), 1,10-phenanthroline and 2,2′-bipyridyl. Preference is given to using TMEDA.

Complex ligand and Cu(II) salt are used in an approximately equimolar ratio. The molar proportion of complex is approximately 0.1 to 10, particularly 1 to 5, preferably approximately 2.5 mol %, based on the carbaldehyde used (XI).

In particular, the decarbonylation is carried out, in addition, in the presence of an organic base. The base used, for example, in the method according to the invention is selected from diazabicycloalkanes such as, for example, diazabicyclooctane (DABCO), diazabicycloundecene (DBU), diazabicyclononane (DBN), tertiary amines such as trimethylamine, triethylamine, diisopropylethylamine or tripropylamine, N,N-dimethylpiperazine, N-methylpyridine, N-methylpyrrolidone, quinuclidine and the like. Preference is given to using DBU. The base is used here in a proportion of 0.1-1 equivalents, 0.2-0,8 equivalents or particularly preferably 0.4-0.6 equivalents, based on the carbaldehydes of formula (XI) used.

The temperature in the reaction is, depending on reactants and solvent used, for example, from 20 to 100° C., such as in particular from 30 to 80° C., particularly from 40 to 60° C. It is also possible to carry out the reaction at two or more temperatures or in two or more temperature ranges which are in each case within the limits specified above. Temperature changes in the course of the reaction may be implemented continuously or discontinuously. However, the reaction temperature is in particular essentially constant.

The pressure during the reaction of the carbaldehydes (XI) with oxygen is particularly ambient pressure or approximately in the range of the autogenous pressure of the reactant or product mixture at the selected reaction temperature(s). The pressure is, for example, from 0.1 to 5 bar, such as from 0.1 to 3 bar, preferably about 1 bar.

It is possible to carry out the reaction at two or more pressures or in two or more pressure ranges which are in each case within the limits specified above. Pressure changes in the course of the reaction may be implemented continuously or discontinuously. However, the pressure during the reaction is in particular essentially constant.

In particular, the reaction may be carried out in batch mode or in continuous mode.

With regard to the reaction vessels which can be used for the reaction (on a laboratory or production scale), there are no particular limitations. In the case of using a reactor, customary stirred reactors, CSTR (continuous stirred tank reactor), in each case with or without internal and/or external heat exchangers, for example a tubular reactor, a tube bundle reactor or a loop reactor may be used. It is also possible to configure the reactors such that it has at least two different zones. Such zones may differ in reaction conditions such as, for example, the temperature or the pressure and/or in the geometry of the zone such as, for example, the volume or the cross section. If the reaction is carried out in two or more reactors, two or more identical reactor types or at least two different reactor types may be used. In particular, however, the reaction is carried out in a single reactor, particularly a stirred reactor.

The residence time of the reaction mixture in the reactor is generally in the range from 0.1 to 40 hours, preferably in the range from 0.1 to 30 hours, more preferably in the range from 0.1 to 25 hours, e.g. in particular 1 to 4 hours.

In particular, the reaction can be carried out as follows;

A reaction mixture consisting of an excess of (E/Z)-cyclohexadec-8-enone and an amount of cyclopentadec-8-enyl carbaldehyde present in deficit, is optionally dissolved in DMF. To the mixture is added as base diazabicycloundecene (DBU, 0.5 to 1.5, e.g. 0.6 eq based on carbaldehyde (XI)). A stream of air was passed continuously through the mixture. The bidentate complex ligand TMEDA (1 to 5 mol %, e.g. 2 mol % based on carbaldehyde (XI)) and Cu(OAc)₂ (1 to 5 mol %, 2 mol %) are dissolved in DMF. Alternatively a Cu-TMEDA mixture can also be prepared without the addition of a solvent. The Cu-TMEDA mixture, with or without DMF, is then added dropwise continuously or in one batch to the reaction solution. The reaction was stirred at 40 to 60, e.g. 50° C. for 10 to 30 h, e.g. 20 h. EtOAc and water is then added. The aqueous phase is adjusted to pH 4 e.g. by means of acetic acid or 98% H₂SO₄. The organic phase is extracted. The aqueous phase is then optionally washed again with EtOAc. The combined organic phases are dried, filtered and concentrated under vacuum.

The residue is then optionally further processed. For example, a fractional distillation is carried out. The cyclopentadecenone X according to the invention can thus be separated from unreacted cyclohexadec-8-enone. The cyclopentadecenone X can then optionally be separated from low boilers, which were not removed by distillation, by column chromatography. For this purpose, for example, silica gel as stationary phase and a mixture of cyclohexane: EtOAc, for example by elution using a stepwise gradient 100:1/30:1/20:1, as mobile phase are suitable. The product is eluted at around 80:1.

The invention is elucidated in detail with reference to the non-limiting working examples below:

### Experimental Section

### Methods:

### Gas Chromatography (GC)

Separating column: CP-Wax 52CB 25 m×0.32 mm×1.2 μm, 1 ml/min N₂

Conditions: 90°-5 min-10°/min-240°-30 min Inj/Det 200°/250° (Method A)

Conditions: 80°-3°/min-250°-Inj/Det 200°/250° (Method B) (Example 2 only)

Sample volumes: 0.2 ml

### GG/MS

Separating column: CP-Wax 52 CB (1.2 μm film thickness), split ratio 10:1

Conditions: 80°-3 min-240°-30 min 0.2 μl

MS conditions: 25-785 amu, 70 eV

### GC/IR

Detector: MCT/A wavelength 650-4000 cm⁻¹

Cells/transfer line temperature 250° C.

Scan 6

Resolution 8

### Column Chromatography

A glass column with frit base was used. The column was packed to ⅔ with swollen silica gel F₂₅₄. The solvent mixture was forced through the column using a positive pressure of 0.2 to 0.4 bar.

Unless Defined Otherwise, “(E/Z)-cyclopentadec-8-enone” is Understood to Mean a Mixture Consisting of (E/Z)-cyclopentadec-8-enone (I) and/or (III) and Regioisomers Thereof (II) and/or (IV).

### Example 1: Preparation of a Cyclohexadecenone/Cyclopentadecenone Mixture According to the Invention

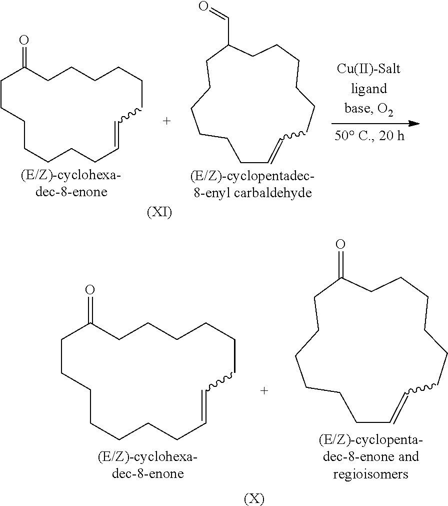

100 g of a mixture consisting of 81% (E/Z)-cyclohexadec-8-enone (Globanone) and 5.9% cyclopentadec-8-enyl carbaldehyde (e.g. prepared according to PCT/EP2015/072544 of the applicant) were dissolved in 300 ml of DMF. To the mixture, 2.3 g of diazabicycloundecene (DBU, 15.2 mmol, 0.6 eq) were added. A stream of air was passed continuously through the mixture. TMEDA (120 mg, 0.5 mmol, 2 mol %) and Cu(OAc)₂ (90 mg, 0.5 mmol, 2 mol %) were dissolved in 30 ml of DMF. The Cu-TMEDA mixture was added dropwise continuously over 6 h via a syringe pump or in one batch to the reaction solution. The reaction was stirred at 50° C. for 20 h. 300 ml of EtOAc were then added and 200 ml of water. The aqueous phase is adjusted to pH 4 by means of 98% H₂SO₄. The organic phase was extracted. The aqueous phase was then washed once again with EtOAc (2×100 ml). The combined organic phases were dried over Na₂SO₄, filtered and concentrated under vacuum. The conversion of the carbaldehydes was >95%. The selectivity of the cyclopentadecenones was 95%. Cyclohexadec-8-enone was not affected.

The residue was worked up by means of fractional distillation in a spinning band column with 20 theoretical plates at 1 mbar top pressure and 125-129° C. head temperature and 170-180° C. bottom temperature. The reflux ratio was 75. The cyclopentadec-7/8-enones were separated from cyclohexadec-8-enone (yield 85%). The cyclopentadec-7/8-enones were subsequently separated from low boilers, which could not be removed in the distillation, by column chromatography (silica gel, mobile phase cyclohexane: EtOAc 100:1, 30:1, 20:1), such that 1 g of a colorless oil was obtained with a purity of 84% (3.7 mmol).

Proportion:
(E)-cyclopentadec-8-enone (I)=54%
(Z)-cyclopentadec-8-enone (III)=30% regioisomer of (I)=0.45%

¹H NMR (500 MHz, CDCl₃, 25° C.): σ=5.3 (m, 2H), 2.5-2.3 (m, 4H), 2.1-1.9 (m, 4H), 1.7-1.55 (m, 4H), 1.45-1.10 (m, 12H) (of the mixture)

### trans-cyclopentadec-8-enone I

¹³C-NMR (125 MHz, CDCl₃, 25° C.): σ=211.98 (C═O), 130.99 (C═C), 41.49 (2×CH₂), 31.80 (2×CH₂), 28.26 (2×CH₂), 28.21 (2×CH₂), 26.84 (2×CH₂), 26.83 (2×CH₂), 22.91 (2×CH₂).

### cis-cyclopentadec-8-enone III

¹³C-NMR (125 MHz, CDCl₃, 25° C.): σ=211.93 (C═O), 130.44 (C═C), 41.57 (2×CH₂), 31.80 (2×CH₂), 28.48 (2×CH₂), 27.68 (2×CH₂), 25.74 (2×CH₂), 23.16 (2×CH₂).

MS m/z=222, 204, 179, 165, 152, 135, 125, 111, 98, 81, 67, 55, 41,

### trans-cyclopentadec-8-enone I

IR (ATR) υ [cm⁻¹]=3022, 2934, 2864, 1724, 1449, 1356, 1121, 969,

### cis-cyclopentadec-8-enone III

IR (ATR) υ [cm⁻¹]=3011, 2935, 2866, 1723, 1456, 1354, 716.

In the GC, a further compound could be found in the mixture whose mass and IR spectrum corresponds to trans-cyclopentadecenone II. The fraction was 0.45%. However, insufficient material was available for a spectroscopic evaluation. The compound could be identified by GC/MS-IR:

### trans-cyclopentadecenone II

IR (ATR) υ [cm⁻¹]=3020, 2934, 2864, 1723, 1449, 1353, 969.

Compound IV could also be identified from a further reaction batch (prepared in accordance with PCT/EP2015/072544 of the Applicant; therein synthesis route 2 or Example 5 by GC/MS-IR:

### cis-cyclopentadecenone IV

IR (ATR) υ [cm⁻¹]=3012, 2936, 2866, 1723, 1457, 1353, 714.

### Example 2: Olfactory Evaluation of a Cyclohexadecenone/Cyclopentadecenone Mixture According to the Invention

Smell test: green, aldehydic, harsh, gassy, musk-like (84% cyclopentadec-8-enone)

| Smell test | Finding |
|---|---|
| Smelling strips <1 min | green, aldehydic, metallic, musk-like |
| Smelling strips 10 min | green, aldehydic, metallic, gassy, musk-like |
| Smelling strips 30 min | green, aldehydic, metallic, gassy, musk-like |
| Smelling strips 1 h | green, aldehydic, harsh, gassy, musk-like |
| Smelling strips 24 h | green, aldehydic, harsh, gassy, musk-like |

### Example 3: Preparation of a Cyclohexadecenone/Cyclopentadecenone Mixture According to the Invention

970 g of a mixture of (E/Z)-cyclohexadec-8-enone (Globanone, 84%) and cyclopent-8-enyl carbaldehyde (XI, 11.5% by weight, 0.52 mol) were dissolved in 944 g of DMF. To the solution, 47.3 g of DBU (0.31 mol, 0.6 eq) were added and the mixture was heated to 50° C. A stream of air was passed continuously through the solution. Using a spray pump in a period of 6 h, 4.7 g of Cu(AcO)₂ (25.9 mmol, 5 mol %) and 3.01 g of TMEDA (25.9 mmol, 5 mol %) dissolved in 150 ml of DMF were added dropwise to the solution. The solution was stirred in total for 20 h at 50° C. 500 ml of water and 8 ml of 98% H₂SO₄ (pH 5) were then added and 500 ml of EtOAc. The phases were stirred vigorously for 10 min and separated. The aqueous phase was then extracted again with 500 ml of EtOAc. The combined organic phases were washed with sat. NaHCO₃ solution, dried over Na₂SO₄, filtered and concentrated. 969 g of a dark brown residue comprising Globanone (83%), (E/Z)-cyclopentadec-8-enone (I, III, 11.5%) and regioisomers of I and III (0.3%) were obtained. This was purified by distillation.

### Example 4: Purifying Distillation of (E/Z)-Cyclopentadec-8-Enone (I, III) and the Regioisomers Thereof

Firstly, the reaction output from Example 3 (969 g of (E/Z)-cyclopentadec-8-enone and the regioisomers thereof (11.8%)) weight)) was separated from high boilers by means of short-path evaporation at 240° C. in the heat transfer oil of the evaporator surfaces and 5 mbar. The distillate was then separated from the low boilers by batch distillation. The number of theoretical plates of the Sulzer DX fabric packing used was 50. This was operated at a top pressure of 10 mbar with a differential pressure across the column of 8 mbar. For the separation of the (E/Z)cyclopentadec-7/8-enone, the reflux ratio was 50. The cyclopentadec-718-enone were fractionally distilled. After the separation, the reflux ratio for the distillation of cyclohexadec-8-enone was reduced to 20. The bottom temperature rose from 204 to 220° C. during the distillation. The top temperature rose from 160 to 175° C.

By means of the fractional distillation, the purity of (E/Z)-cyclopentadec-8-enone and the regioisomers thereof, see above, could be increased from 11.7% to 94-98% (area %). The ratio of trans- to cis-cyclopentadec-8-enone here was 4:1 (Fr. 3) and 2:1 (Fr. 4).

### Example 5: Olfactory Classification of the 98% Cyclopentadecenone Sample

Samples “Fr. 3” and “Fr. 4” from Example 4

| | |
|---|---|
| Intensity | 4 |
| Musk | 6 |
| Sweet, powdery | 4 |
| Dry fruit | 2 |

### Example 6: Preparation of a Cyclohexadecenone/Cyclopentadecenone Mixture According to the Invention in the Absence of a Solvent

18.5 g of diazabicycloundecene (DBU, 121.8 mmol, 0.6 eq), 0.6 g of TMEDA (5.1 mmol, 2.5 mol %) and 0.92 g of Cu(OAc)₂ (5.1 mmol, 2.5 mol %) were added to 400 g of a mixture consisting of 81% (E/Z)-cyclohexadec-8-enone and 5.9% cyclopentadec-8-enyl carbaldehyde (e.g. prepared in accordance with PCT/EP2015/072544 of the applicant). The reaction mixture was heated to 50° C. and a stream of air (100 NI/h) was passed continuously through the mixture. The mixture was stirred for 4 h. Subsequently, 200 ml of EtOAc, 200 ml of water and 43.4 g of AcOH were added at room temperature and the mixture was stirred for 5 min. The phases were separated and the aqueous phase was extracted once again with 200 ml of EtOAc. The combined organic phases were dried over Na₂SO₄, filtered and concentrated. The conversion of the carbaldehydes was 97%. The selectivity for cyclopentadecenones was 85%. The loss of cyclohexadec-8-enone was only 0.2%.

The disclosure of the publications mentioned herein is explicitly incorporated by reference.

1.-20. (canceled)

21. A method for preparing a macrocyclic keto compound of formula X 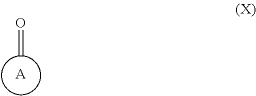
wherein A is a cycloaliphatic hydrocarbon residue having m ring carbon atoms, where m is an integer from 13 to 17, and optionally has n C═C double bonds, where n is an integer equal to 1, 2 or 3, the method comprising)
a) Oxidatively decarbonylating a cycloaliphatic carbaldehyde compound of formula XI 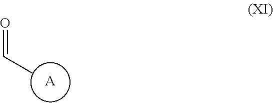
where A is as defined above; wherein the compound of formula XI is reacted in the presence of a Cu(II) catalyst and molecular oxygen; and optionally
b) Isolating at least one compound of formula X from the reaction mixture.

22. The method as claimed in claim 21, wherein the catalyst is formed in situ by adding a ligand to a Cu(II) salt.

23. The method as claimed in claim 21, wherein m is 15 and/or n is 1 in the compounds of formula X and XI.

24. The method as claimed in claim 21, wherein (E/Z)-cyclopentadec-8-enyl-1-carbaldehyde or a mixture of (E/Z)-cyclopentadec-8-enyl- and (E/Z)-cyclopentadec-7-enyl-1-carbaldehyde, or a mixture comprising this compound, is used.

25. The method as claimed in claim 24, wherein at least one compound is obtained selected from the group consisting of (E/Z)-cyclopentadec-8-en-1-one and the regioisomers thereof.

26. The method as claimed in claim 25, wherein at least one compound is obtained selected from compounds of formulae I and III and regioisomers thereof of formulae II and IV

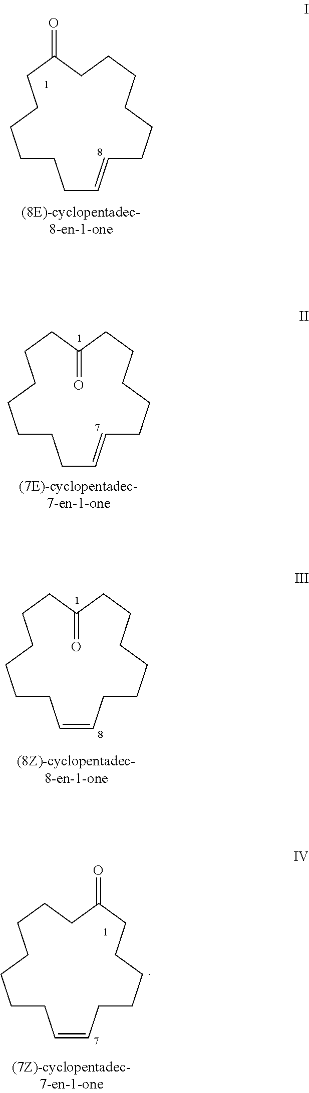 [SMILES: O=C1CCCCCC/C=C/CCCCCC1 ; O=C1CCCCC/C=C/CCCCCCC1 ; O=C1CCCCCC/C=C\CCCCCC1 ; O=C1CCCCC/C=C\CCCCCCC1]

27. The method as claimed in claim 21, wherein a reaction product is used in stage a) obtained from the dinitrogen monoxide oxidation of a cycloaliphatic compound of formula XII 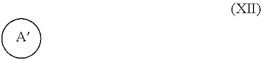
where A′ is a cycloaliphatic hydrocarbon residue having m+1 ring carbon atoms, where m is an integer from 13 to 17, and optionally has n+1 C═C double bonds, and where n is an integer equal to 1, 2 or 3.

28. The method as claimed in claim 27, wherein m is 15 and n is 1 in the compound of formula XII.

29. The method as claimed in claim 28, wherein the compound of formula XII is cyclohexadeca-1,9-diene.

30. A method for preparing Globanone ((E/Z)-cyclohexadec-8-en-1-one), comprising
a) oxidizing cyclohexadeca-1,9-diene with dinitrogen monoxide, wherein a reaction mixture is obtained, which comprises a mixture comprising Globanone and at least one cyclopentadecenyl carbaldehyde compound, particularly selected from (E/Z)Application cyclopentadec-8-enyl-1-carbaldehydes; wherein unreacted cyclohexadeca-1,9-diene is optionally removed;
b) subjecting the reaction mixture from stage a) to an oxidative decarbonylation reaction as claimed in claim 21; and
c) separating Globanone from the cyclopentadecenones I thus formed.

31. The method as claimed in claim 21, wherein the oxidative decarbonylation is carried out in the presence or absence of a solvent.

32. A substance or substance mixture comprising at least one macrocyclic keto compound of formula X 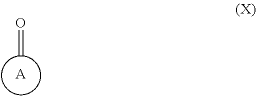
wherein A is a cycloaliphatic hydrocarbon residue having m ring carbon atoms, where m is an integer from 13 to 17, and optionally has n C═C double bonds, where n is an integer equal to 1, 2 or 3.

33. The substance mixture as claimed in claim 32 comprising at least two compounds selected from the group consisting of (E/Z)-cyclopentadec-8-en-1-one and the regioisomers thereof.

34. The substance mixture as claimed in claim 33 comprising at least two compounds selected from compounds of formulae I and III and regioisomers thereof of formula II: 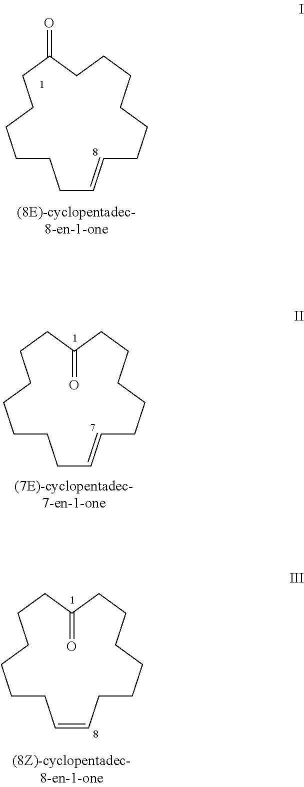 [SMILES: O=C1CCCCCC/C=C/CCCCCC1 ; O=C1CCCCC/C=C/CCCCCCC1 ; O=C1CCCCCC/C=C\CCCCCC1]
or comprising at least two compounds selected from the group consisting of compounds of the following formulae I and III and optionally regioisomers thereof of the formulae II and IV

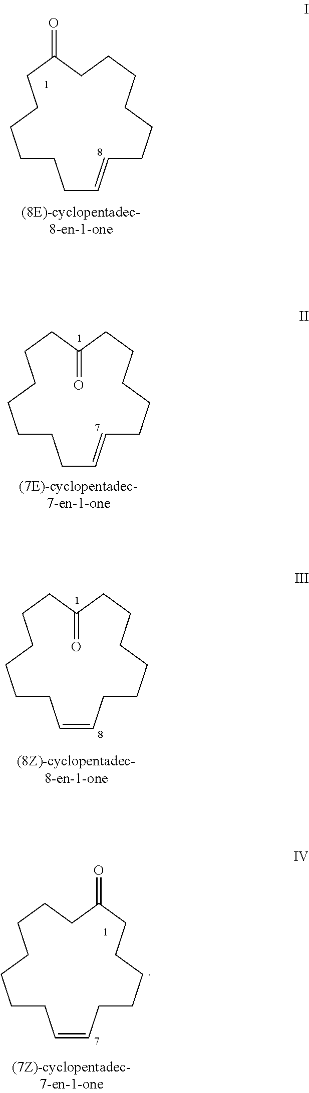 [SMILES: O=C1CCCCCC/C=C/CCCCCC1 ; O=C1CCCCC/C=C/CCCCCCC1 ; O=C1CCCCCC/C=C\CCCCCC1 ; O=C1CCCCC/C=C\CCCCCCC1]

35. A fragrance composition comprising at least one substance or substance mixture as claimed in claim 31 as an aroma chemical.

36. The composition as claimed in claim 35 of a mixture essentially comprising (E)cyclopentadec-8-en-1-one (I) and (Z)-cyclopentadec-8-en-1-one (III).

37. The composition as claimed in claim 36, wherein the molar ratio of (I) to (III) is in the range of approximately 1:1 to approximately 5:1.

38. The composition as claimed in claim 35 wherein the aroma chemical generates a musk note in the fragrance composition.

39. A fragrance composition comprising at least one substance or one substance mixture prepared according to the method as claimed in claim 21.

40. An agent comprising at least one substance or one substance mixture according to the definition in claim 32.
